(19) United States
(12) Patent Application Publication
(10) Pub. No.: US 2011/0269838 A1
(43) Pub. Date: Nov. 3, 2011

(54) NOVEL PROCESSES AND PURE POLYMORPHS

(76) Inventors: Vinayak Gore, Maharashtra (IN); Madhukar Patil, Maharashtra (IN); Rahul Bhalerao, Maharashtra (IN); Hemant Mande, Maharashtra (IN)

(21) Appl. No.: 13/131,238

(22) PCT Filed: Nov. 25, 2009
(86) PCT No.: PCT/GB2009/051597

§ 371 Date: Jul. 12, 2011

(30) Foreign Application Priority Data
Nov. 26, 2008 (IN) 2056/KOL/2008

(51) Int. Cl. A61K 31/167; A61P 35/00; C07C 259/06

(52) U.S. Cl. 514/575; 562/623

(57) ABSTRACT

The present invention relates to crystalline forms of the active pharmaceutical ingredient vorinostat, processes for their preparation and their use in pharmaceutical compositions.

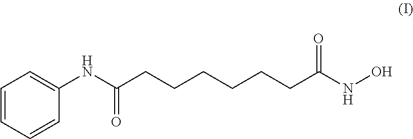

### FIELD OF THE INVENTION

The present invention relates to crystalline forms of the active pharmaceutical ingredient vorinostat, processes for their preparation and their use in pharmaceutical compositions.

### BACKGROUND OF THE INVENTION

The manufacturing process for many pharmaceuticals is hindered by the fact that the organic compound, which is the active ingredient, has handling difficulties during the manufacturing process and may impart undesirable properties to the final drug or dosage form. In addition it can be difficult to control the polymorphic form of the active pharmaceutical ingredient throughout the manufacturing process.

For pharmaceuticals in which the active ingredient can exist in more than one polymorphic form, it is particularly important to ensure that the manufacturing process for the active ingredient affords a single, pure polymorph with a consistent level of polymorphic purity. If the manufacturing process leads to a polymorph with varying degrees of polymorphic purity and/or where the process does not control polymorphic interconversion, serious problems in dissolution and/or bioavailability can result in the finished pharmaceutical composition comprising the active ingredient.

Vorinostat, represented by structural formula (I) and chemically named as N-hydroxy-N′-phenyl-octanediamide or suberoylanilide hydroxamic acid (SAHA), is a member of a larger class of compounds that inhibit histone deacetylases (HDAC). Histone deacetylase inhibitors (HDI) have a broad spectrum of epigenetic activities and vorinostat is marketed, under the brand name Zolinza®, for the treatment of a type of skin cancer called cutaneous T-cell lymphoma (CTCL). Vorinostat is approved to be used when the disease persists, gets worse, or comes back during or after treatment with other medicines. Vorinostat has also been used to treat Sézary's disease and, in addition, possesses some activity against recurrent glioblastoma multiforme.

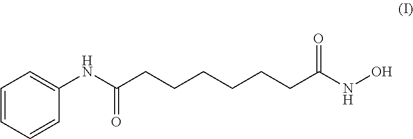

Vorinostat was first described in U.S. Pat. No. 5,369,108, but no polymorphic data was mentioned. Five crystalline forms of vorinostat, designated forms I to V respectively, were disclosed in documents US 2004/0122101 and WO 2006/127319. However, the five forms disclosed in these documents suffer from several disadvantages which do not make them ideal forms for pharmaceutical development. The prior art processes to prepare forms I to V, and in particular form I, suffer from the disadvantages of being inconsistent and difficult to reproduce, and they produce polymorphically impure products. The prior art processes are particularly inconvenient for large scale production.

If crystalline forms are made with polymorphic impurities, this causes instability and it can accelerate significant interconversion to another polymorphic form. Therefore it is crucial to produce crystalline forms with very high polymorphic purity to avoid this interconversion.

In view of the importance acquired by vorinostat for the treatment of cancer, there is a great need for developing an alternative, relatively simple, economical and commercially feasible process for the synthesis of vorinostat crystalline forms with commercially acceptable yield, high polymorphic stability and polymorphic purity.

### OBJECT OF THE INVENTION

Therefore an object of the present invention is to improve existing processes to prepare known polymorphs of vorinostat and to improve the polymorphic purity of known polymorphs, to provide polymorphic forms of vorinostat which are convenient to manufacture and which have improved properties suitable for formulation development and as a marketed pharmaceutical composition.

### SUMMARY OF THE INVENTION

The present inventors have developed convenient processes for the preparation of crystalline vorinostat form I, which are consistent, very reproducible and convenient for large scale production, and which produce form I in very high polymorphic purity.

The present inventors have surprisingly developed new processes to polymorphically pure crystalline vorinostat form I, which circumvent the problems associated with the processes reported in the prior art as described above, in particular inconvenience for large scale production and low polymorphic purity.

As used herein throughout the specification and claims, the term “vorinostat form I” refers to the vorinostat form I as described and characterised in US 2004/0122101 and WO 2006/127319. Preferably the vorinostat form I is characterised by an XRPD spectrum comprising three or more (preferably four or more, preferably five or more, preferably six or more, preferably seven or more, preferably eight or more, preferably nine or more, preferably ten or more, or preferably all eleven) of the following degrees 2θ peaks: 9.0, 9.4, 17.5, 19.4, 20.0, 24.0, 24.4, 24.8, 25.0, 28.0, 43.3±0.2 degrees 2θ, when measured using a Siemens D500 Automated Powder Diffractometer equipped with a copper radiation source with a wavelength of 1.54 Å. Preferably the vorinostat form I is characterised by a differential scanning calorimetry (DSC) trace with an endothermic peak at about 164.3° C.±2.0° C., when measured using a Perkin Elmer instrument, over a temperature range of from 50° C. to 30° C. above the observed melting temperature, at a heating rate of 10° C./min, using a standard aluminium pan and cover as crucible.

Therefore, in a first aspect of the present invention there is provided a process for the preparation of crystalline vorinostat form I comprising: dissolving vorinostat in an organic solvent; mixing the solution formed with water; and isolating the form I formed from the mixture.

Preferably, the solution is mixed with water by pouring it into water. Preferably, the solution to be mixed with water is a clear solution.

Preferably, in the process according to the first aspect of the present invention, the organic solvent is selected from an alcohol, an amide and mixtures thereof. Preferred amides are N,N-dimethylformamide and N,N-dimethylacetamide; a more preferred amide is N,N-dimethylformamide. Preferred alcohols are methanol, ethanol, isopropanol, 1-butanol, 2-butanol and tert-butanol; more preferred alcohols are methanol, ethanol and isopropanol; a more preferred alcohol is methanol. Preferably, the organic solvent is selected from methanol, ethanol, N,N-dimethylformamide, isopropanol, 1-butanol, 2-butanol, tert-butanol and mixtures thereof.

Preferably, for each gram of vorinostat about 2-10 ml of organic solvent are used, preferably about 4-6 ml of organic solvent, preferably about 5 ml of organic solvent.

Preferably, in the process according to the first aspect of the present invention, the organic solvent is heated between 40 to 100° C. to dissolve the vorinostat, more preferably at about 60° C. Preferably, the vorinostat is completely dissolved in the organic solvent to form a clear solution.

Preferably, in the process according to the first aspect of the present invention, the water acts an anti-solvent causing the vorinostat to precipitate out of solution.

If an amide is used as organic solvent, preferably for each gram of vorinostat about 10-50 ml of water are used, preferably about 20-30 ml of water, preferably about 25 ml of water. If an alcohol is used as organic solvent, preferably for each gram of vorinostat about 2-10 ml of water are used, preferably about 4-6 ml of water, preferably about 5 ml of water.

Preferably, in the process according to the first aspect of the present invention, the mixture is cooled before isolation of the crystalline vorinostat form I. Preferably, the mixture is cooled to between 5 to 30° C., preferably to between 25 to 30° C., more preferably to about 25° C.

Preferably, in the process according to the first aspect of the present invention, the vorinostat form I is isolated by filtration. Preferably, the isolated vorinostat form I is dried, preferably at about 60° C., preferably under vacuum, preferably for about 2-10 hours, more preferably for about 5-6 hours, preferably until a constant weight is achieved.

Preferably, in the process according to the first aspect of the present invention, the process is carried out without milling. Preferably, in the process according to the first aspect of the present invention, the process is carried out without seeding.

In a second aspect of the present invention there is provided a process for the preparation of crystalline vorinostat form I comprising: dissolving vorinostat in water; and isolating the form I from the mixture.

Preferably, for each gram of vorinostat about 2-10 ml of water are used, preferably about 4-6 ml of water, preferably about 5 ml of water.

Preferably, in the process according to the second aspect of the present invention, the water is heated to between 40 to 100° C. to dissolve the vorinostat, more preferably to about 60° C. Preferably, the vorinostat is completely dissolved in the water to form a clear solution.

Preferably, in the process according to the second aspect of the present invention, the water is cooled to afford the crystalline vorinostat form I. Preferably, the water is cooled to between 5 to 30° C., preferably to between 25 to 30° C., more preferably to about 25° C.

Preferably, in the process according to the second aspect of the present invention, the vorinostat form I is isolated by filtration. Preferably, the isolated vorinostat form I is dried, preferably at about 60° C., preferably under vacuum, preferably for about 2-10 hours, preferably until a constant weight is achieved.

Preferably, in the process according to the second aspect of the present invention, the process is carried out without milling. Preferably, in the process according to the second aspect of the present invention, the process is carried out without seeding.

In a third aspect of the present invention there is provided a process for the preparation of crystalline vorinostat form I comprising: dissolving vorinostat in a first organic solvent; mixing the solution formed with a second organic solvent; and isolating the form I formed in the mixture.

Preferably, the solution is mixed with a second organic solvent by pouring it into the second organic solvent. Preferably, the solution to be mixed with the second organic solvent is a clear solution.

Preferably, in the process according to the third aspect of the present invention, the first organic solvent is selected from an alcohol, an amide and mixtures thereof. Preferably, the first organic solvent is selected from methanol, ethanol, N,N-dimethylformamide and mixtures thereof.

Preferably, for each gram of vorinostat about 2-10 ml of first organic solvent are used, preferably about 4-6 ml of first organic solvent, preferably about 5 ml of first organic solvent.

Preferably, in the process according to the third aspect of the present invention, the first organic solvent is heated between 40 to 100° C. to dissolve the vorinostat, more preferably at about 60° C. Preferably, the vorinostat is completely dissolved in the first organic solvent to form a clear solution.

Preferably, in the process according to the third aspect of the present invention, the second organic solvent is selected from a ketone, a nitrile, an ester and mixtures thereof.

Preferably, the second organic solvent is selected from acetone, methyl ethyl ketone, methyl isobutyl ketone, diethyl ketone, acetonitrile, propionitrile, ethyl acetate, methyl acetate and mixtures thereof.

Preferably, in the process according to the third aspect of the present invention, the second organic solvent acts an anti-solvent causing the vorinostat to precipitate out of solution.

Preferably, for each gram of vorinostat about 2-10 ml of second organic solvent are used, preferably about 4-6 ml of second organic solvent, preferably about 5 ml of second organic solvent.

Preferably, in the process according to the third aspect of the present invention, the mixture is cooled before isolation of the crystalline vorinostat form I. Preferably, the mixture is cooled to between 5 to 30° C., preferably to between 25 to 30° C., more preferably to about 25° C.

Preferably, in the process according to the third aspect of the present invention, the vorinostat form I is isolated by filtration. Preferably, the isolated vorinostat form I is dried, preferably at about 60° C., preferably under vacuum, preferably for about 2-10 hours, preferably until a constant weight is achieved.

Preferably, in the process according to the third aspect of the present invention, the process is carried out without milling. Preferably, in the process according to the third aspect of the present invention, the process is carried out without seeding.

In a fourth aspect of the present invention there is provided crystalline vorinostat form I, as prepared by a process according to the first, second or third aspect of the present invention.

Preferably, the crystalline vorinostat form I according to fourth aspect of the present invention comprises less than 2% of vorinostat in other polymorphic forms, preferably less than 1%, more preferably less than 0.5%, even more preferably less than 0.2%, and most preferably less than 0.1%, preferably as measured by XRPD or DSC, preferably as measured by XRPD.

In a fifth aspect of the present invention there is provided crystalline vorinostat form I, comprising less than 2% of vorinostat in other polymorphic forms, preferably comprising less than 1% of vorinostat in other polymorphic forms, more preferably comprising less than 0.5% of vorinostat in other polymorphic forms, even more preferably comprising less than 0.2% of vorinostat in other polymorphic forms, and most preferably comprising less than 0.1% of vorinostat in other polymorphic forms, preferably as measured by XRPD or DSC, preferably as measured by XRPD.

Preferably, the crystalline vorinostat form I according to the fourth or fifth aspect of the present invention has a chemical purity of at least 95%, more preferably at least 98%, more preferably at least 99%, more preferably at least 99.5%, even more preferably at least 99.8%, and most preferably at least 99.9%, preferably as measured by HPLC.

Preferably, the crystalline vorinostat form I according to the fourth or fifth aspect of the present invention is suitable for use in medicine, preferably for treating cancer, preferably for treating skin cancer, preferably for treating cutaneous T-cell lymphoma (CTCL).

In a sixth aspect of the present invention there is provided a pharmaceutical composition, comprising the crystalline vorinostat form I according to the fourth or fifth aspect of the present invention. Preferably, the pharmaceutical composition further comprises one or more pharmaceutically acceptable excipients. Preferably, the pharmaceutical composition is for treating cancer, preferably for treating skin cancer, preferably for treating cutaneous T-cell lymphoma (CTCL).

In a seventh aspect of the present invention there is provided the use of the crystalline vorinostat form I according to the fourth or fifth aspect of the present invention or the use of the pharmaceutical composition according to the sixth aspect of the present invention, in the manufacture of a medicament for the treatment of cancer, preferably for the treatment of skin cancer, more preferably for the treatment of cutaneous T-cell lymphoma (CTCL).

In an eighth aspect of the present invention there is provided a method of treating cancer, comprising administering to a patient in need thereof a therapeutically effective amount of the crystalline vorinostat form I according to the fourth or fifth aspect of the present invention or a therapeutically effective amount of the pharmaceutical composition according to the sixth aspect of the present invention. Preferably, the method is for the treatment of skin cancer, more preferably for the treatment of cutaneous T-cell lymphoma (CTCL). Preferably, the patient is a mammal, preferably a human.

### DETAILED DESCRIPTION OF THE INVENTION

As outlined above, the present invention provides convenient processes for the preparation of crystalline vorinostat form I which avoid the problems associated with prior art processes.

These processes use mild conditions and temperatures, thus minimizing the occurrence of polymorphic interconversion and producing form I with very high polymorphic purity and stability, which avoids the problems associated with the prior art form I.

Preferred embodiments of the processes according to the present invention are described below.

Preferred processes for the preparation of crystalline vorinostat form I can use, as starting material, any prior art form of vorinostat, including crystalline forms I to V of vorinostat disclosed in US 2004/0122101 and WO 2006/127319, or the novel crystalline form VI of vorinostat as reported in a co-pending application by the present inventors (Indian patent application IN 2057/KOL/2008 and the international patent application claiming priority therefrom).

A particularly preferred embodiment of the process according to the first aspect of the present invention comprises dissolving vorinostat in an organic solvent at about 60° C., pouring the clear solution formed into water, cooling the mixture and filtering the crystalline vorinostat form I formed.

Preferably, the organic solvent, in the process according to the first aspect of the present invention, is selected from methanol, ethanol, N,N-dimethylformamide, isopropanol, 1-butanol, 2-butanol, tert-butanol and mixtures thereof.

A particularly preferred embodiment of the process according to the second aspect of the present invention comprises dissolving vorinostat in water by heating at about 60° C. for about 1 hour. The clear solution is allowed to cool to about 25° C. before isolating the crystalline vorinostat form I from the mixture by filtration.

A particularly preferred embodiment of the process according to the third aspect of the present invention comprises dissolving vorinostat in a first organic solvent at about 60° C., and then pouring the clear solution formed into a second organic solvent, followed by cooling the mixture to about 25° C. and filtering the crystalline vorinostat form I formed.

Preferably, the first organic solvent, in the process according to the third aspect of the present invention, is selected from methanol, ethanol, N,N-dimethylformamide and mixtures thereof.

Preferably, the second organic solvent, in the process according to the third aspect of the present invention, is selected from acetone, methyl ethyl ketone, methyl isobutyl ketone, diethyl ketone, acetonitrile, propionitrile, ethyl acetate, methyl acetate and mixtures thereof.

Preferably, the crystalline vorinostat form I, obtained by the processes according to the first, second and third aspects of the present invention, is dried until the moisture content falls below about 1%, preferably to below about 0.5%.

The major advantages of this invention are milder and reproducible experimental conditions of the processes of the present invention to obtain the polymorph and the polymorphic purity and stability of the crystalline form I obtained.

The pharmaceutical composition according to the sixth aspect of the present invention can be a solution or a suspension, but is preferably a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant and optionally further comprises at least one excipient selected from colouring agents, adsorbents, surfactants, film-formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments and fillers.

Preferably, the pharmaceutical compositions according to the sixth aspect of the invention are for use in the treatment of cancer, preferably in the treatment of skin cancer, and more preferably in the treatment of cutaneous T-cell lymphoma (CTCL).

Preferably, the pharmaceutical compositions according to the present invention are in unit dosage form comprising vorinostat in an amount of from 1 mg to 500 mg, such that the amount of vorinostat administered is from 0.1 mg to 100 mg per kg per day.

The details of the invention, its objects and advantages are illustrated below in greater detail by non-limiting examples.

### EXAMPLES

### Example 1

### Preparation of Crystalline Vorinostat Form I

Vorinostat (10 g) was charged to a reaction flask containing amide (50 ml) (organic solvent). The resulting suspension was heated at 60° C. for one hour under stirring. The resulting clear solution was poured into water (250 ml) at 20-25° C. White solid precipitated out. The solid product was filtered and dried at 60° C. under vacuum until a constant weight was obtained.

| | | Chemical |
|---|---|---|
| Amide used | Yield | purity (as measured by HPLC) |
| N,N-dimethylformamide | 8.0 g (80%) | ≧99.9% |
| N,N-dimethylacetamide | 8.1 g (81%) | ≧99.9% |

### Example 2

### Preparation of Crystalline Vorinostat Form I

Vorinostat (10 g) was charged to a reaction flask containing alcohol (50 ml) (organic solvent). The resulting suspension was heated at 60° C. for one hour under stirring. The resulting clear solution was poured into water (50 ml) at 20-25° C. White solid precipitated out. The solid product was filtered and dried at 60° C. under vacuum until a constant weight was obtained.

| Alcohol used | Yield | Chemical purity (as measured by HPLC) |
|---|---|---|
| methanol | 8.5 g (85%) | ≧99.9% |
| ethanol | 6.7 g (67%) | ≧99.9% |
| isopropanol | 7.1 g (71%) | ≧99.9% |
| 1-butanol | 7.3 g (73%) | ≧99.9% |
| 2-butanol | 7.6 g (76%) | ≧99.9% |
| tert-butanol | 7.5 g (75%) | ≧99.9% |

### Example 3

### Preparation of Crystalline Vorinostat Form I

Vorinostat (10 g) was charged to a reaction flask containing methanol (50 ml) (organic solvent). The suspension was heated at 60° C. for one hour under stirring. The resulting clear solution was poured into water (50-300 ml, typically 50 ml when the organic solvent is an alcohol, typically 250 ml when the organic solvent is an amide). The reaction mixture was cooled to 25° C. and filtered. The solid product obtained was dried at 60° C. under vacuum until a constant weight was obtained.

Chemical purity ≧99.9% (as measured by HPLC)

The above procedure in example 3 was repeated using different organic solvents to obtain vorinostat form I, namely:

Organic solvent: methanol, ethanol, isopropanol, 1-butanol, 2-butanol, tert-butanol, N,N-dimethylformamide, N,N-dimethylacetamide.

### Example 4

### Preparation of Crystalline Vorinostat Form I

Vorinostat (10 g) was charged to a reaction flask containing water (50 ml). The resulting mixture was heated for one hour at 60° C. to obtain a clear solution. The reaction mixture was cooled to 25° C. and it was filtered. The solid product was dried at 60° C. under vacuum until a constant weight was obtained.

Chemical purity ≧99.9% (as measured by HPLC)

### Example 5

### Preparation of Crystalline Vorinostat Form I

Vorinostat (10 g) was charged to a reaction flask containing N,N-dimethylformamide (50 ml) (organic solvent I). The resulting suspension was heated at 60° C. for one hour under stirring. The resulting clear solution was poured into acetone (50 ml) (organic solvent II). The reaction mixture was cooled to 25° C. and it was filtered. The solid product was dried at 60° C. under vacuum until a constant weight was obtained.

Chemical purity ≧99.9% (as measured by HPLC)

The above procedure in example 5 was repeated using different solvents to obtain vorinostat form I, namely:

Organic solvent I: methanol, ethanol, N,N-dimethylformamide.

Organic solvent II: acetone, methyl ethyl ketone, methyl isobutyl ketone, diethyl ketone, acetonitrile, propionitrile, ethyl acetate, methyl acetate.

Each organic solvent I listed above was used with each organic solvent II listed above to prepare vorinostat form I.

In all of examples 1 to 5, ¹H-NMR indicated formation of vorinostat. XRPD and DSC analysis data confirmed that the products obtained were crystalline form I of vorinostat, in accordance with the data disclosed in US 2004/0122101 and WO 2006/127319.

The samples of crystalline vorinostat form I prepared in the above examples were found to be substantially pure polymorphically with no levels of other forms detected (>99.7% polymorphically pure), as measured by XRPD and DSC. The samples of crystalline vorinostat form I prepared were also found to be very stable polymorphically with no conversion over time to other polymorphs, when kept at a temperature of 40° C.±2° C. and a relative humidity of 75%±5% for 6 months.

It will be understood that the present invention has been described above by way of example only. The examples are not intended to limit the scope of the invention. Various modifications and embodiments can be made without departing from the scope and spirit of the invention, which is defined by the following claims only.

1-22. (canceled)

23. A process for the preparation of crystalline vorinostat form I comprising: dissolving vorinostat in an organic solvent; mixing the solution formed with water; and isolating the form I formed from the mixture.

24. The process according to claim 23, wherein the organic solvent is selected from methanol, ethanol, isopropanol, 1-butanol, 2-butanol, tert-butanol, N,N-dimethylformamide, N,N-dimethylacetamide or a mixture thereof.

25. The process according to claim 23, wherein the organic solvent is heated between 40 to 100° C. to dissolve the vorinostat.

26. The process according to claim 25, wherein the organic solvent is heated at about 60° C.

27. The process according to claim 23, wherein the mixture is cooled before isolation of the crystalline vorinostat form I.

28. The process according to claim 27, wherein the mixture is cooled to between 5 to 30° C.

29. The process according to claim 28, wherein the mixture is cooled to about 25° C.

30. Crystalline vorinostat form I, as prepared by a process according to claim 23.

31. Crystalline vorinostat form I, comprising less than 2% of vorinostat in other polymorphic forms.

32. The crystalline vorinostat form I according to claim 30 for:
(i) use in medicine; and/or
(ii) treating cancer; and/or
(iii) treating skin cancer; and/or
(iv) treating cutaneous T-cell lymphoma (CTCL).

33. The crystalline vorinostat form I according to claim 31 for:
(i) use in medicine; and/or
(ii) treating cancer; and/or
(iii) treating skin cancer; and/or
(iv) treating cutaneous T-cell lymphoma (CTCL).

34. A pharmaceutical composition, comprising crystalline vorinostat form I according to claim 30 and one or more pharmaceutically acceptable excipients.

35. A pharmaceutical composition, comprising crystalline vorinostat form I according to claim 31 and one or more pharmaceutically acceptable excipients.

36. The pharmaceutical composition according to claim 34 for treating:
(i) cancer; and/or
(ii) skin cancer; and/or
(iii) cutaneous T-cell lymphoma (CTCL).

37. The pharmaceutical composition according to claim 35 for treating:
(i) cancer; and/or
(ii) skin cancer; and/or
(iii) cutaneous T-cell lymphoma (CTCL).

38. A method of treating cancer, comprising administering to a patient in need thereof a therapeutically effective amount of the crystalline vorinostat form I according claim 30.

39. A method of treating cancer, comprising administering to a patient in need thereof a therapeutically effective amount of the crystalline vorinostat form I according claim 31.

40. A method of treating cancer, comprising administering to a patient in need thereof a therapeutically effective amount of the pharmaceutical composition according to claim 34.

41. A method of treating cancer, comprising administering to a patient in need thereof a therapeutically effective amount of the pharmaceutical composition according to claim 35.

42. The method according to claim 38, wherein:
(i) the method is for the treatment of skin cancer; and/or
(ii) the method is for the treatment of cutaneous T-cell lymphoma (CTCL); and/or
(iii) the patient is a mammal; and/or
(iv) the patient is a human.

43. The method according to claim 39, wherein:
(i) the method is for the treatment of skin cancer; and/or
(ii) the method is for the treatment of cutaneous T-cell lymphoma (CTCL); and/or
(iii) the patient is a mammal; and/or
(iv) the patient is a human.

44. The method according to claim 40, wherein:
(i) the method is for the treatment of skin cancer; and/or
(ii) the method is for the treatment of cutaneous T-cell lymphoma (CTCL); and/or
(iii) the patient is a mammal; and/or
(iv) the patient is a human.

45. The method according to claim 41, wherein:
(i) the method is for the treatment of skin cancer; and/or
(ii) the method is for the treatment of cutaneous T-cell lymphoma (CTCL); and/or
(iii) the patient is a mammal; and/or
(iv) the patient is a human.
